(19) United States
(12) Patent Application Publication
(10) Pub. No.: US 2024/0344022 A1
(43) Pub. Date: Oct. 17, 2024

(54) ACTIVE TISSUE SCAFFOLD STRETCHING UNDER CELL CULTURE CONDITIONS

(71) Applicant: ARIZONA BOARD OF REGENTS ON BEHALF OF THE UNIVERSITY OF ARIZONA, Tucson, AZ (US)

(72) Inventors: Frederic ZENHAUSERN, Phoenix, AZ (US); Alan NORDQUIST, Phoenix, AZ (US); Baiju THOMAS, Phoenix, AZ (US); Matthew BARRETT, Phoenix, AZ (US); Jerome LACOMBE, Phoenix, AZ (US); Ashlee HARRIS, Phoenix, AZ (US)

(21) Appl. No.: 18/294,371

(22) PCT Filed: Aug. 1, 2022
(86) PCT No.: PCT/US22/39017

§ 371 Date: Feb. 1, 2024

Related U.S. Application Data
(60) Provisional application No. 63/228,436, filed on Aug. 2, 2021.

(51) Int. Cl. C12N 5/00; C12M 1/12; C12M 1/42

(52) U.S. Cl. CPC C12N 5/0062; C12M 25/14; C12M 35/04; C12N 2513/00; C12N 2533/90

(57) ABSTRACT

Provided herein are stretch devices and related methods for controlled three-dimensional stretching of scaffolds supported on a stretchable substrate. A stretchable substrate is capable of receiving the three-dimensional scaffold, and a stretchable substrate holder is configured to connect to the stretchable substrate. An actuator is connected to the stretchable substrate holder to exert a cyclic stretch on the stretchable substrate and periodically stretch the three-dimensional scaffold connected to the stretchable substrate. A sample holder is configured to immerse the three-dimensional scaffold in a tissue culture media.

### CROSS-REFERENCE TO RELATED APPLICATIONS

This application claims the benefit of priority of U.S. Provisional Patent App. No. 63/228,436, filed Aug. 2, 2021, which is hereby incorporated by reference in its entirety.

### BACKGROUND

The use of porous 3D scaffolds to provide a suitable environment for the generation of tissues and organs is vital for the exploration of novel cellular models for biomedical research. For these purposes, several biomaterials have been explored including ceramics, metals, bioactive glasses, animal-derived tissues, polymers, etc., and more recently, plants and plant-based polymers have also emerged as relevant biomaterials. Plant-based scaffolds have many practical advantages including the apparent ease with which they can be made and manipulated; they are quite pliable and can be easily cut, fashioned, rolled or stacked to form a range of different sizes and shapes. From physical and architectural perspectives, plant tissues have promising properties, including high surface area, interconnected porosity, natural vascular networks, various range of stiffness and mechanical properties, and excellent water transport and retention. Finally, from a biochemical perceptive, plant-based scaffolds are mainly made of cellulose, a biocompatible and nonimmunogenic material that allows cell adhesion and can have pro-angiogenic function in vivo. What governs some of the intrinsic cellular behavior and molecular functions in tissue and organs are complex interactions between biochemical and physical clues, in particular when related to mechanical effects. There is a need in the art for cell culture tools that can reliably provide dynamic mechanical stimuli to cells or tissue constructs on compliant scaffolds, including polymers, cellular assemblies and/or plant-based biomaterials.

Although exerting cyclic stretch on cultured cells is known (see, e.g., Carosi et al. “Cyclical strain effects on production of vasoactive materials in cultured endothelial cells” J Cell Physiol. 1992 April: 151 (1): 29-36), those systems are inherently 2D, with cells cultured directly on an elastic membrane and, upon culture to confluence, the elastic membrane upon which the cells are directly supported is stretched. The devices and methods provided herein are fundamentally different and address the problem of exerting a cyclic stretch on a cellular system that is more realistically provided in a 3D geometry.

### BRIEF SUMMARY

Provided herein are devices and related methods for applying a controlled dynamic force on a macroscale tissue scaffold that is compatible with cell culture conditions that can range from minutes to weeks. The devices and methods address the fundamental problem of exerting physiologically relevant forces, such as cyclic stretch, on a geometrically relevant cellular system by providing biological cells in a three-dimensional scaffold which, in turn, is supported by a stretchable substrate, which receives a cyclic stretch from an actuator. The devices are compatible for integration into a culture dish system or well plate device containing all the necessary media and components for culture in an environment such as a CO₂ thermo-incubator. The apparatus is also made of materials compatible with various sterilization techniques, i.e. autoclaving, supercritical fluid CO₂, UV illumination, X-ray or gamma ray irradiation. Exemplary materials can comprise polymers, hard plastics, rubbers or other flexible materials, all biocompatible and known to not induce any cellular toxicity.

A mechanically compliant material is used as a substrate onto which a tissue scaffold (also referred herein as a “three-dimensional scaffold” or “3D scaffold”) is attached. Exemplary substrates comprise polymeric rubber, cellulosic film, thin piezoelectric materials, bi-metallic memory materials or any other extendable and compressible substrate that is biocompatible with the organic biomaterials of interest such as cells or tissues. The substrate may be described as a stretchable substrate in that the substrate can be strained under an applied force, including in elongation and/or compression, and return to a resting state once the applied force is removed. The substrates may be coated with a biocompatible layer, including a layer comprising proteins or other biologically relevant materials.

Actuators are used to provide a well-controlled time-varying force on the stretchable substrate. For example, mechanical actuation with a mechanical actuator may comprise an electrical actuator delivering a translation motion of the substrate frame with the scaffold through a rotational cam and gear box, or by using liquid compression and release through a fluidic activation by using a combination of syringes and pumps, an electromagnetic actuation or application of a vacuum. The devices and methods provided herein are compatible with any number of actuator types and any of a range of relevant actuation described in the prior art of mechanics and electronics may be used.

The devices and methods provided herein may be used to modulate a desired biological effect by exerting a desired mechanical force (e.g., stretching magnitude, frequency, strain rate). One example of a biological effect that can be modulated by mechanical forces includes tissue fibrosis. Other molecular effects are detectable by changes in gene expression, protein expression, metabolites secretion, mutations, cellular microvesicles release or other morphological or phenotypic manifestation.

Provided herein is a device for stretching a three-dimensional scaffold under cell culture conditions comprising a stretchable substrate for receiving the three-dimensional scaffold and a stretchable substrate holder configured to connect to the stretchable substrate. An actuator is operably connected to the stretchable substrate holder to exert a cyclic stretch on the stretchable substrate and, thereby, periodically stretch the three-dimensional scaffold connected to the stretchable substrate. A sample holder configured to immerse the three-dimensional scaffold in a tissue culture media and expose the three-dimensional scaffold to cell culture parameters ensures ongoing viability of living cells supported by the three-dimensional scaffold. The sample holder may comprise a volume sufficient to immerse the substrates in culture media, even while the substrates are undergoing cyclic stretch. The volume may be accessible to supplement, refresh and/or change culture media, so that the cells are receive appropriate nutrients and physical conditions (pH, salts) for healthy conditions.

The three-dimensional scaffold may comprise a natural scaffold or an artificial scaffold. Preferable natural scaffold includes plant tissue, such as corresponding to a decellularized plant. Artificial scaffolds may comprise polymers, elastomers, rubbers or other materials that mimic physical properties of tissue while being biocompatible with biological cells.

The stretchable substrate may comprise a polymer, a cellulosic film, a thin piezoelectric material, a bi-metallic memory material, or a decellularized tissue.

A biocompatible layer may be positioned between the stretchable substrate and the three-dimensional scaffold, so that attachment between the substrate and scaffold is enhanced in a biocompatible manner that does not adversely impact biological cells supported by the scaffold.

The actuator may comprise any number of components useful to provide controlled stretch, including a power supply, a cam, and a gearbox operably connected to generate a mechanically driven translational motion of a portion of the stretchable substrate holder connected to the stretchable substrate.

To facilitate controlled motion, the actuator may comprise one or more of an electrical motor, an electrical vacuum pump, a fluidic pump, and/or an electromagnetic actuator.

The actuator may be a uniaxial, bi-axial, multi-axial or radial actuator. This can be achieved by connecting multiple actuators at different locations to stretch the substrate in different directions. Alternatively, a pressure difference may be exerted across the substrate surface, causing the entire surface of the substrate to deform, with a maximum stretch in a central region and minimum at the edges connected to the holder.

The actuator may provide a stretch up to 100%, including up to 20% stretch, for a time period up to 2 months under the cell culture parameters. Depending on the application of interest, appropriate average strains (e.g., 4% to 30%) are utilized over a desired time period (minutes to days).

The device may further comprise an electronic controller connected to the actuator to provide a user-specified stretch protocol, wherein the user-specified stretch protocol includes a maximum strain magnitude, strain rate and/or time-course,

The device may further comprise a power source that is a battery.

The device may further comprise a cell culture controller to control the cell culture parameters around the stretchable substrate, including cell culture parameters that are temperature between 32° C. and 40° C., a CO₂ level of between 4% and 6%, an O₂ level of between 2% and 21%, and/or a humidity of between 80% and 98%.

The device may have a device geometric parameter configured to position at least the stretchable substrate and three-dimensional scaffold into a cell culture incubator that provides the cell culture parameters. In this manner, the incubator controls may control the cell culture conditions.

The device may further comprise a sensor for monitoring one or more of the cell culture parameters. In this manner, an alert or alarm may indicate when one or more parameters are out of range.

The three-dimensional scaffold may comprise a decellularized plant tissue, including one that supports a cell culture or transplanted cells.

Also provided is a method of periodically stretching a three-dimensional scaffold using any of the devices described herein. For example, the method may comprise the steps of connecting a stretchable substrate to the stretchable substrate holder of any of the devices described herein and attaching the three-dimensional scaffold to the stretchable substrate. A periodic force is exerted on the stretchable substrate by actuating the actuator, thereby periodically stretching the three-dimensional scaffold.

The exerting step may be for a time period ranging from minutes to weeks.

The method may further comprise the step of culturing one or more biological cells supported by the three-dimensional scaffold.

The actuating step may be selected to produce a desired biological response parameter from the one or more biological cells, the response parameter selected from the group consisting of: gene expression; protein expression; metabolite secretion; mutation; cellular microvesicle release; phenotype expression; alignment, and cell proliferation. In other words, a suitable strain, strain rate, frequency and duration can be selected depending on the desired biological response parameter.

The three-dimensional scaffold may comprise a decellularized scaffold from a vegetal or an animal tissue (e.g., extracellular matrix such as collagen, elastin and/or glycosaminoglycan); artificial scaffold made with a synthetic material (e.g. silicon, polylactic-coglycolic acid, polyurethane, poly(glycerolsebacate), polyacrylamide, etc.) or natural material (e.g. plant protein (e.g. soy, zein, wheat glutenin, etc.), plant polysaccharides (e.g. cellulose, pectin, starch, etc.), lignin, plant extracts, alginate, collagen, gelatin, hyaluronan, fibrin, chitosan, etc.); or a polymer.

The actuating step may comprise a maximum bulk stretch of the three-dimensional scaffold of up to 100%, including between 5% and 25%, at a cyclic stretch of between 1 cycle per minute and 200 cycles per minute.

The stretchable substrate may have a uniform stretch distribution and the three-dimensional scaffold may have a spatially varying stretch distribution. For example, a region of the stretchable substrate may have a less than 20% maximum variation from an average stretch whereas the scaffold may have a greater than 20% maximum variation. The materials may be selected to provide a desired maximum variation, such as between 5% and 30%.

The method may further comprise the step of controlling a periodic force to obtain a desired biological response of biological cells cultured in the periodically stretched three-dimensional scaffold.

Also provided herein is a method of making any of the devices described herein by providing each of the components and arranging them as described to achieve the desired three-dimensional scaffold stretching.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWING(S)

FIGS. 1A-1B are photographs of an assembled device for stretching a three-dimensional tissue scaffold, including under cell culture conditions. Exemplary components include a stretchable substrate to support tissue scaffold, holder for attaching the scaffold, actuator with controlled motion for moving holder to stretch and compress stretchable substrate, and a cell culture dish (sample holder). FIG. 1C is an exploded view of the device of FIG. 1B to further illustrate the various components, including an actuator, off-centered CAM, motor stage, springs, slide mechanism, scaffold clamping system (in this embodiment, the scaffold is a leaf scaffold), and medium reservoir. In this manner, periodic stretching (as exemplified in FIG. 6) is achieved. FIG. 1D is another device schematic.

FIG. 2: is a schematic of the device of FIG. 1A, providing representative dimensions of the different components for securing the tissue scaffold, and providing reliable operable connection between motor and strain exerted on the stretchable components.

FIGS. 3A-3D illustrate device development, corresponding to first through fourth iterations, respectively, to achieve an end goal of a cost-effective system capable of long term culturing and stretching.

FIG. 4 summarizes a design configuration for the device, including how the motor can effect a translational motion of the stretchable substrate and attached scaffold to provide periodic stretching to the substrate and scaffold. The top left panel summarizes the various devices used to generate the controllable forces and corresponding stretch, including the motor, CAM, platform, glider and reservoir (in which the substrate and scaffold can be immersed) for living biological cell applications. The motor shaft turns a rounded cam that pushes the glider to produce the elongation.

FIG. 5 is a summary of the method of obtaining a 3D scaffold from a vegetation tissue (e.g., a leaf), culturing cells with the 3D scaffold, applying stretch for a given period and, as desired, evaluating the effects of the stretch, including cyclic stretch.

FIG. 6A schematic illustration of a cyclic stretch having a longitudinal maximum strain of about 13% a frequency of 0.31 Hz, and a strain rate of 7.6%/second, matching the physiological range of lung elongation during breathing. Due to the Poisson effect (FIG. 6B), there is a corresponding lateral strain, expressed as an overall y-axis strain in a direction normal to the longitudinal strain (x-axis strain).

FIG. 7A: Characterization Protocol: Several dots were placed on the biomaterial (cut in the shape of a typical leaf scaffold) equidistantly spaced between the two clamps in the clamping system. Then, videos of the biomaterial stretching (taken at 1080p resolution and 30 frames per second) were processed using imaging software, and analyzed using Image J to measure the displacement of each dot from its original position during the stretch. FIG. 7B Strain distribution results in x and y directions for each of stretchable substrate (PDMS) and three-dimensional tissue scaffold (leaf).

FIG. 8A are florescent images for control (no stretch-left panel) and cyclic stretched (right panel) illustrating a biological response to mechanical stretch. Stretching induces A549 cells to align perpendicular to the direction of stretch. Immunofluorescence images of F-Actin (green) and nuclei (blue) in A549 cells seeded on leaf scaffolds which were either stretched or static (control) in the custom stretch device for 24 hrs. FIGS. 8B-8C illustrate that in control, cells stained for F-actin display random orientation and alignment. Yet, A549 cells exposed to the stretch were found to orient themselves more perpendicular to the stretch direction. This strain-induced re-orientation has been previously shown as the cells adapt to their changing environment by aligning against the stretch to alleviate the stress on their fibers. Mean nuclear orientation angle for cells on unstretched (control) and stretched scaffolds (FIG. 8B). A minimum of 100 total cells were utilized to determine the nuclear angles for each experimental group. Distribution of nuclear angles in the control and stretched experimental groups (FIG. 8C).

FIG. 9A-9C: YAP localizes to nucleus in response to mechanical strain. FIG. 9A YAP (yes-associated protein) is the major mechanotransduction signaling pathway of the cell where cells physically perceive their microenvironment and translate that stimuli into biochemical signals that ultimately control cell behavior, growth, differentiation, etc. On soft substrates, such as decellularized leaf scaffolds, YAP is inactive and in the cytoplasm. However, on stiff substrates, YAP is activated and translocated to the nucleus. Thus, in the un-stretched control, YAP is primarily found in the cytosol. However, under mechanical strain, YAP is shifted into the nucleus. FIG. 9B Quantification of nuclear positive YAP on the stretched scaffolds. FIG. 9C CTGF, a target gene of YAP was found to be overexpressed compared to the control due to the activation of YAP as a transcription factor.

FIG. 10A-10C: Gene expression is modified in response to mechanical strain. Quantitative RT-PCR analysis of gene expression in A549 cells on stretched and unstretched controls. FIG. 10A Four types of collagen were found to be overexpressed in response to mechanical stress. FIG. 10B Mechanotransduction membrane ion channels Piezo1 and Piezo2 were shown to be underexpressed. FIG. 10C Known cytokine interleukin-8 (IL-8) was also overexpressed compared to control. While these findings are consistent with the known results from the literature, investigation of the behavioral response of cells stretched on the decellularized leaf scaffolds is ongoing.

FIG. 11: Close up view of scaffold and stretchable substrate clamped and ready to undergo cyclic stretch via the actuator.

FIG. 12: Flow-chart summary of method of making a three-dimensional scaffold from plant, with subsequent culturing, cyclic stretch and characterization/testing of the cells supported by the 3D scaffold.

FIG. 13A-13C are plots summarizing mechanical testing (strain at failure—FIG. 13A) and ultimate tensile strength (UTS)—FIG. 13B) and water retention (FIG. 13C) for control and stretch conditions.

FIG. 14A-14C summarize localized strain in a 3×3 grid for longitudinal direction (FIG. 14A), lateral direction (FIG. 14B) for PDMS (left panels), decellularized leaf (middle panels) and decellularized lung (right panels). FIG. 14C is a plot of longitudinal (left panel) and lateral (right panel) strain variability (see FIG. 6B for directional definition.

FIG. 15A-15I illustrate cells seeded on decellularized plant scaffold, in this case spinach leaf, sense and respond to mechanical strain, as evidenced by fluorescent microscopy (FIG. 15A), nuclear orientation and size (FIGS. 15B-15C), protein expression (FIGS. 9A-9C 15D), mRNA expression (FIG. 15E), calcium level (FIG. 15F), calmodulin (CALM) and collagen gene expression (FIGS. 15G, 15H respectively). Histological stained with Masson's Trichrome illustrates increase in collagen secretion for stretched cells.

FIGS. 16A-16B summarize longitudinal (FIG. 16A) and lateral (FIG. 16A) localized strains of a 3×3 grid on each of PDMS, decellularized leaf and decellularized lung.

### DETAILED DESCRIPTION

The following examples further illustrate the invention but, of course, should not be construed as in any way limiting its scope.

“Scaffold” refers to a material that is capable of supporting living biological cells, including cultured cells under culture conditions, “Three-dimensional” refers to a spatial pattern of the scaffold, including in a manner that models tissue when cultured cells are grown under stretch and cell culture conditions. Accordingly, scaffold may be referred to as a “tissue scaffold.” The scaffold itself may be formed from a biological material (“natural scaffold”) or a synthetic material (“synthetic scaffold”), or a combination thereof (“composite scaffold”), so long as it is able to be supported by the stretchable substrate with attendant cyclic stretch.

“Stretchable substrate” refers to a material that is able to repeatedly stretch without significant hysteresis or damage. Stretch may be defined by a maximum strain, such as Δy/y, where Δy is the magnitude of the deformation, and the stretch or strain may be expressed as percentage. The stretchable substrate may be further defined by a modulus, such as a Young's modulus, Y=stress/strain, and may, depending on the application of interest, be selected from between that range of 100 kPa to 100 GPa.

“Operably connected” refers to a configuration of elements, wherein an action or reaction of one element affects another element, but in a manner that preserves each element's functionality. For example, during use the actuator, including a motor, may be characterized as being “operably connected” to a stretchable substrate, but in a manner that ensures the substrate is not adversely impacted. Elements that are operably connected may be directly connected or indirectly connected via one or more intervening elements. For example, between the actuator and substrate, there may be a substrate holder, such as a clamp, that is in turn connected to a translational rod, connected to a CAM that is connected to a motor of the actuator.

The devices and methods described herein provides means to apply controlled dynamic forces on a macroscale tissue scaffold during a period of cell culture conditions that can range from minutes to weeks. As it is biocompatible, the device is also designed for integration into a culture dish system or well plate device containing all the necessary media and components for culture in an environment such as a CO₂ thermo-incubator. The device is also made of materials compatible with most of the sterilization techniques, i.e. autoclaving, supercritical fluid CO₂, UV illumination, X-ray or gamma ray irradiation. These materials can comprise polymers, hard plastics, rubbers or other flexible materials. Thus, it is re-usable.

There are many challenges limiting the clinical applicability of tissue engineering solutions. As discussed, there is a need for new biomaterials to produce scaffolds that can be used to model in-vivo tissue structures, both mechanically (Young's moduli) and geometrically (3D structure), while being able to support biological cells, including in a culture and under mechanical stress. The devices and methods provided herein utilize decellularized plant tissue, obtained by decellularization of plant materials, such as from a leaf, fruit, vegetable, stem, trunk, and the like, depending on the desired application of interest. Unique cellulose-based plant scaffold features include an innate vasculature, biocompatibility, elasticity, topography and stiffness. Applications include tissue engineering, regenerative medicine, drug delivery systems, and cellular biology modeling.

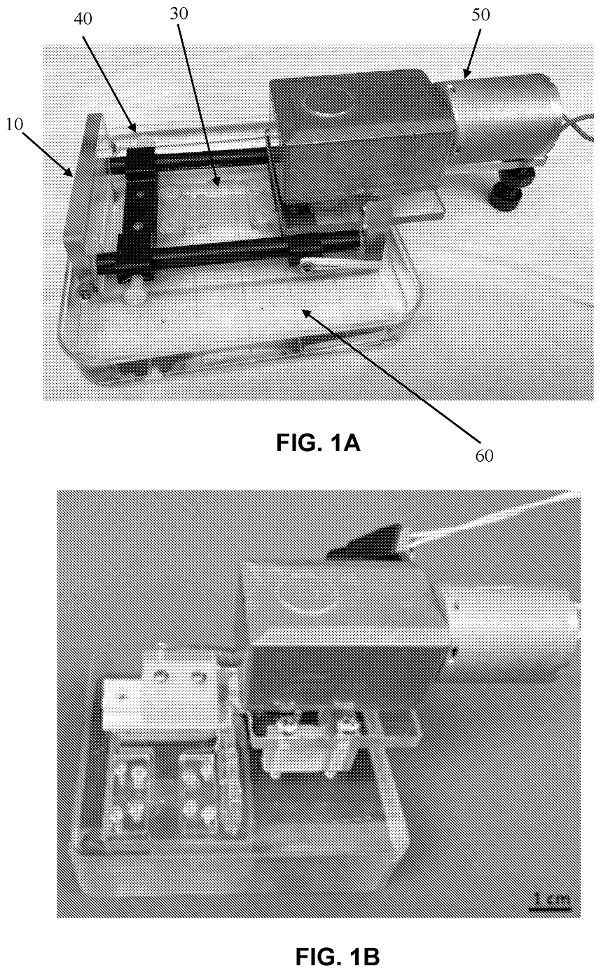

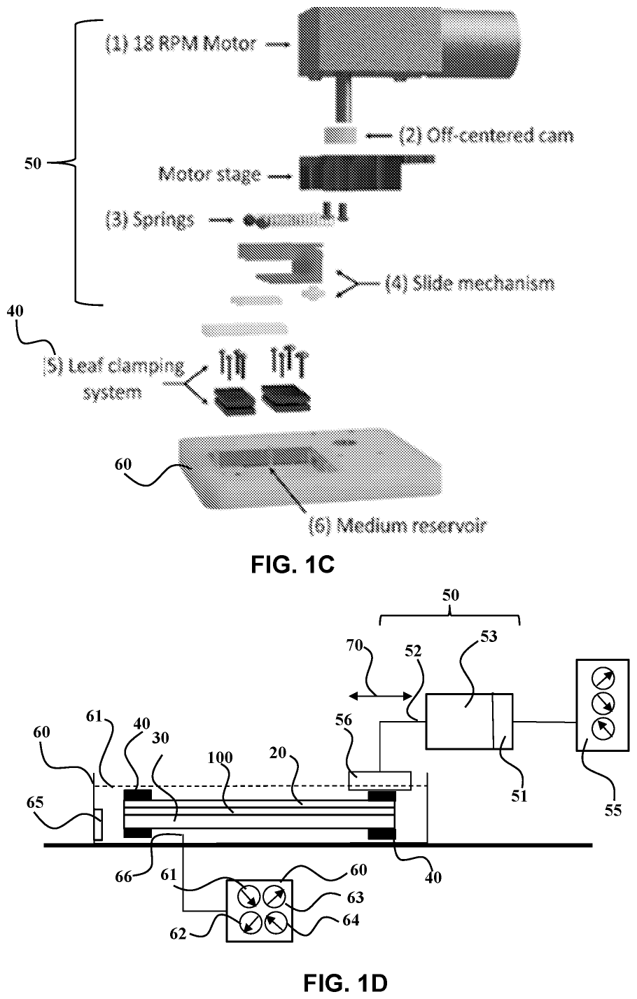

A schematic illustration of a device is provided in FIG. 1D. Stretchable substrate 20 supports a biocompatible layer 100 and a three-dimensional scaffold 30. Stretchable substrate holder 40 reliably holds the substrate 20 and scaffold 30 in position. The holder may comprise clamps at each end of the substrate and scaffold. The holder may clamp only the substrate for aspects where the scaffold is strongly adhered to the substrate with an adhesive force sufficient to resist stresses associated with the cyclic stretch of the substrate. Alternatively, the holder may hold both the substrate and scaffold. The holder may be a fastener to which the substrate and/or scaffold is fastened. The holder may be an adhesive which adhesively binds the substrate and/or scaffold. The holder may be any combination thereof.

Actuator 50 that generates a translational motion 70 is operably connected to the holder 40, such as at one end. By fixing the other end of the holder 40, motion 70 provided by translational motion 56 of, for example, a glider generates the corresponding cyclic stretch of substrate and scaffold corresponding to 70. Actuator may be further described with respect to various components, such as power supply 51, cam 52, gear box 53 (and motor). Electronic controller 55 may be electronically connected to actuator for controlling any one or more stretch parameters, such as magnitude, frequency, and strain rate.

Sample holder 60 may correspond to a volume in which the scaffold 20 is positioned. In this manner, tissue culture media 61 ensures living cells (not shown) supported by scaffold 20 remain healthy. Tissue culture parameters 66, may be controlled by cell culture controller 60, with parameters such as temperature 61, CO₂ level 62, O₂ level 63 and humidity 64, each adjustable. The cell culture controller 60 may be part of an incubator used to cell culture, particularly as the geometric footprint of device 10 lends itself for being placed into a conventional cell culture incubator. Sensor(s) 65 may be used to monitor any one or more cell culture parameters, including in a feedback loop to ensure the parameters remain within a user-selected range.

### Example 1: Device for Stretching 3D Tissue Scaffold

FIGS. 1A-1B are photographs of a device 10 for stretching a three-dimensional tissue scaffold under cell culture conditions. As illustrated, a stretchable substrate 30 is provided onto which a tissue scaffold (three-dimensional tissue scaffold) can be supported or attached. Examples of substrates include polymeric rubber, cellulosic film, thin piezoelectric materials, bi-metallic memory materials, decellularized tissues or any other extendable and compressible substrate that is biocompatible with the organic biomaterials of interest, such as cells or tissues. A biocompatible layer may be provided between the stretchable substrate and the tissue scaffold, including by a layer or coating of a stretchable substrate surface. Coating of these substrates with proteins or other biocompatible layer.

An actuator 50 generates a motion of the stretchable substrate holder 40 so as to provide an associated deformation stretch in stretchable substrate, and attendant stretch of the scaffold. Any of a variety of actuators may be used. For example, mechanical actuation can comprise an electrical actuator delivering a translation motion of the substrate frame with the scaffold through a rotational cam and gear box, or by using liquid compression and release through a fluidic activation by using a combination of syringes and pumps, an electromagnetic actuation or even by applying some vacuum (see, e.g., U.S. Pat. Nos. 4,839,280; 6,048,723) or other relevant actuation described in the prior art of mechanics and electronics. In this manner, substrate 30 experiences controlled motion that stretches and then compresses the substrate. In this manner, the substrate and associated scaffold experiences “cyclic stretch.” Sample holder 60, corresponding functionally to a cell culture dish, holds media appropriate to the scaffold and any living cells or material supported therein.

FIG. 1C is an exploded view of the device 10, illustrating motor, off-centered cam, motor stage, springs, slide mechanism; that together may be considered to form the actuator 50 that provides the motion. Of course, other actuators are compatible with the instant invention, so long as there is a reliable and controlled cyclic stretch, including under cell culture conditions. For example, instead of a cam driving a translational motion, a vacuum pump that controls pressure on one side of an airtight substrate held by substrate holder, can be used to provide a cyclic stretch. The stretchable substrate holder 50 may be a clamping system and the sample holder 60 may have a reservoir in which the scaffold is immersed.

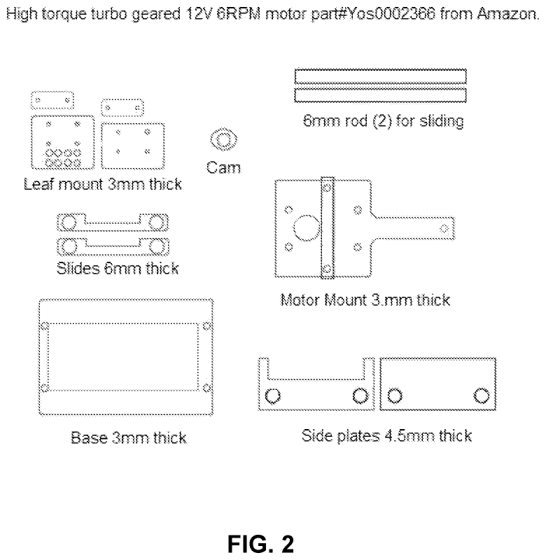

FIG. 2 is a design schematic of various portions of the device illustrated in FIG. 1, including electrical motor, cam rod, mounts, slides, base, and side plates.

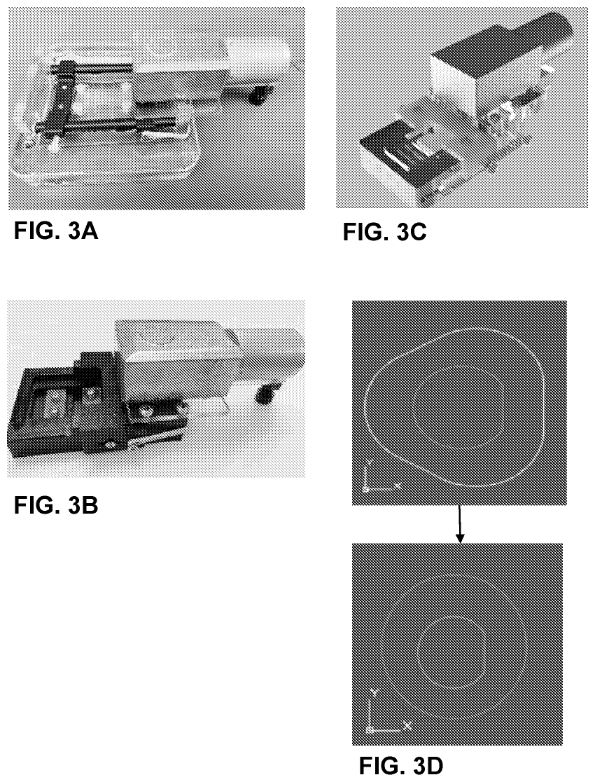

FIGS. 3A-3D illustrate various design iterations. FIG. 3A is a Gen 1 device (open design) that is effective at stretching scaffolds but that required too much media, making use of the system expensive. FIG. 3B is a Gen 2 device (ABS plastic) that utilizes less media (cost effective), but is not biocompatible with A549 cells. FIG. 3C is a Gen 3 device (trilobed CAM) that is biocompatible and effective at clamping, but has the CAM resting in media reservoir, risking contamination and does not always stretch straight. FIG. 3D is a Gen 4 device (single lobed CAM placed higher that is biocompatible; effective clamping of leaf; motor rotates cam which sits out of the medium reservoir as to not cause contamination; plastic glides with minimal friction; stretches straight and level, but that is not an enclosed system.

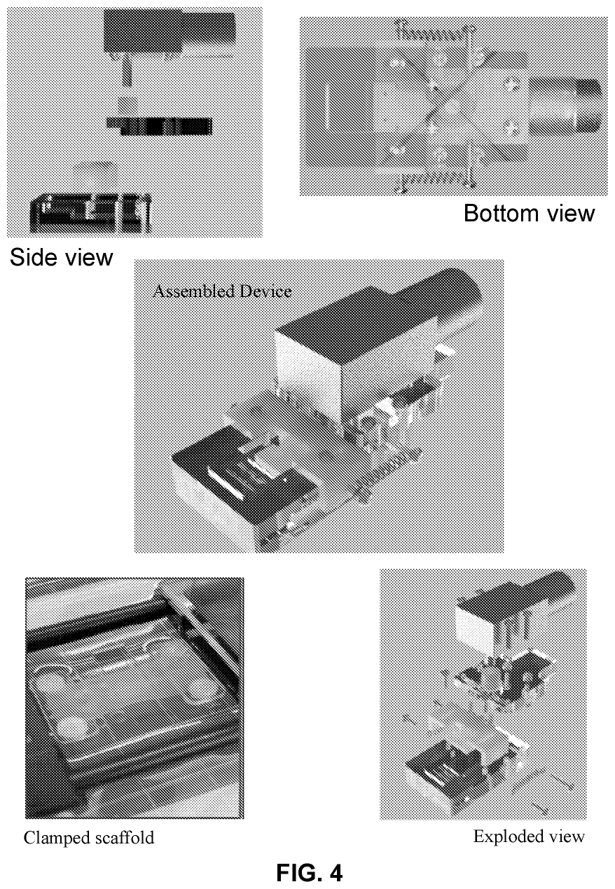

FIG. 4 are photographs of various views of a device, including a close-up view of the clamped scaffold. The actuator comprises an 18 RPM mother shaft that turns a rounded cam which pushes a glider to produce the elongation in a periodic fashion.

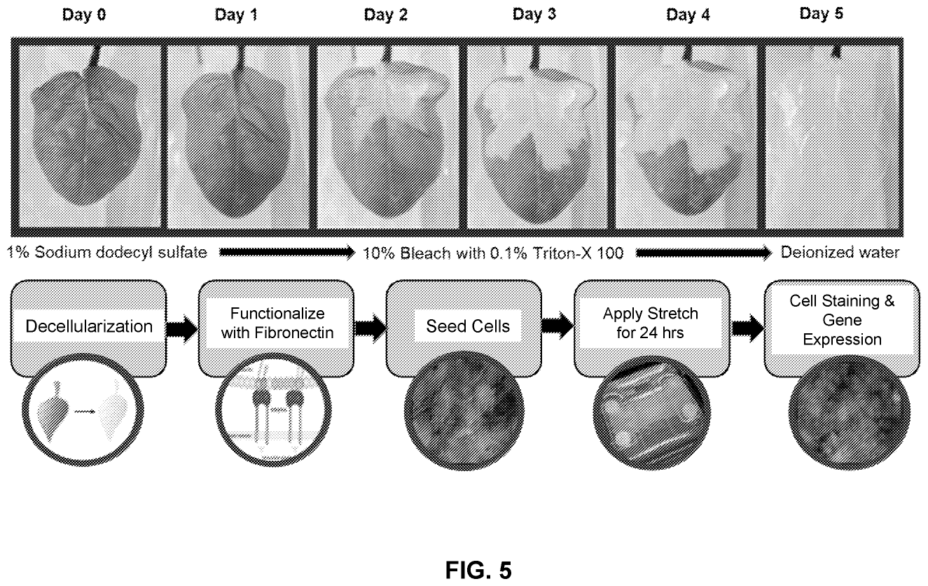

As further explained, data indicates that the device can stretch a cellulose scaffold made from decellularized spinach leaf (see, e.g., FIG. 5) with an average elongation of 13% in the direction of the stretch and a contraction of 5% in the direction perpendicular to the stretch. The local elongation for the leaf differs greatly due to the heterogeneity of the leaf structure. In addition, cells seeded on the stretched leaves are responsive to the stretch initiated by the device. This is further reflected by observation of realignment of the cells in a direction perpendicular to the direction of the stretch, the nuclear location of YAP protein and the overexpression of collagen and interleukin genes.

The devices and methods are compatible with one or more of: lid/enclosed system (also allow for gas exchange); simultaneous stretch of multiple tissues; stretch/elongation from multiple sides (e.g., multi-axial or radial stretch); stretch/elongation from different directions, at different rates, strain, etc. (e.g., stretch controller) depending on the application; incorporation of biosensors; self-sustaining power; cell culture controllers (e.g., temperature control).

The devices and methods are useful for a number and range of applications, including: various and/or multiple cell types; Cell alignment or orientation angle; extracellular matrix (ECM) secretion; Cell surfactant/vesicle secretion; Cell gene expression; Cell protein expression; Cell death pathways (e.g., apoptosis); Cellular ion regulation; pH alterations; Mechanotransduction pathways; Response to external stimuli (e.g. irradiation/drug).

### Example 2: Decellularized Plant Tissue to Model Lung Breathing Mechanical Stress

Despite the remarkable advancements made in tissue engineering (TE), there are still many challenges limiting the clinical applicability of TE solutions, which has prompted the search for new biomaterials. In this example, we utilize as a three-dimensional scaffold decellularized plant materials, to recapitulate the lung microenvironment. Specifically, spinach leaves are utilized as a biocompatible scaffold to model the mechanical stress caused by the lung during breathing. Physiological lung values in a healthy human adult are 12-20 breaths/minute and 10-20% elongation during breathing.

First, an engineered device is developed to stretch the scaffold and mimic physiological breath motions. The characterization data demonstrates that the device can apply a cyclic strain at a frequency of 18 cycles/min with an average strain amplitude of 13%. Lung epithelial A549 cells are then seeded on the decellularized scaffolds that are placed in the stretch device for 24 hours. After stretching, the cells are immunostained for YAP and F-Actin, and the expressed mRNA measured using qRT-PCR. The results indicate: (1) cells align perpendicular to the direction of the stretch, (2) the cytoplasmic YAP protein localizes to the nucleus and (3) collagen type I, IV and VI as well as interleukin-8 mRNA levels increase, indicating that the cells are able to sense and adapt to the mechanical strain. These results indicate the lung-on-a-leaf model is a suitable platform to further study other biological responses in the lung microenvironment.

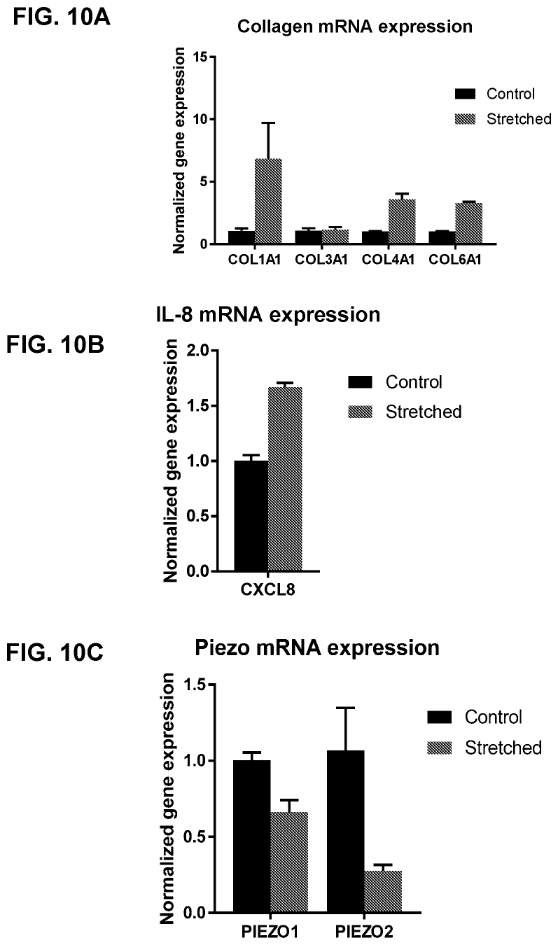

Referring to FIGS. 5-11, this example demonstrates an elastic decellularized plant tissue used with a device of the instant invention, including under cell tissue conditions for growth of seeded cells with the scaffold. Representative physiological properties include mechanical stress on tissue associated with respiration and blood flow, such as on any one or more of the lung, heart, blood vessels. The elastic properties (including specifically Young's modulus) of decellularized plant leaves, such as spinach, that form the 3D scaffold, are manipulated to be about 1 kPa to 30 kPa. This range overlaps with relevant biological systems, including blood vessel walls and lung. The device can be set to match physiological parameters such as 12-20 breaths/minute and 10-20% elongation during breathing.

Processing steps for obtaining a 3-D scaffold from a plant tissue is summarized in FIG. 5. FIG. 5 illustrates a decellularization process and various other aspects associated with methods of using the device. Fibronectin coating: Scaffolds were coated with a μg/mL Fibronectin solution and incubated overnight at 37° C. prior to cell seeding. Cell Seeding: Any desired cells may be seeded, including A549 cells seeded at 250,000 cells/cm² and incubated overnight at 37° C. in a 5% CO₂ atmosphere to promote cell attachment to the scaffold. Stretch experiments: Leaves were clamped in the stretch device and underwent mechanical strain for 24 hours. Cell Staining: Cells were fixed with 4% paraformaldehyde (PFA) in PBS and immunostained with YAP and F-actin. Gene Expression: RNA was extracted using RNAspin Mini kit (GE Healthcare) and 300 ng of total RNA was used to perform a quantitative RT-PCR analysis of gene expression in the A549 cells.

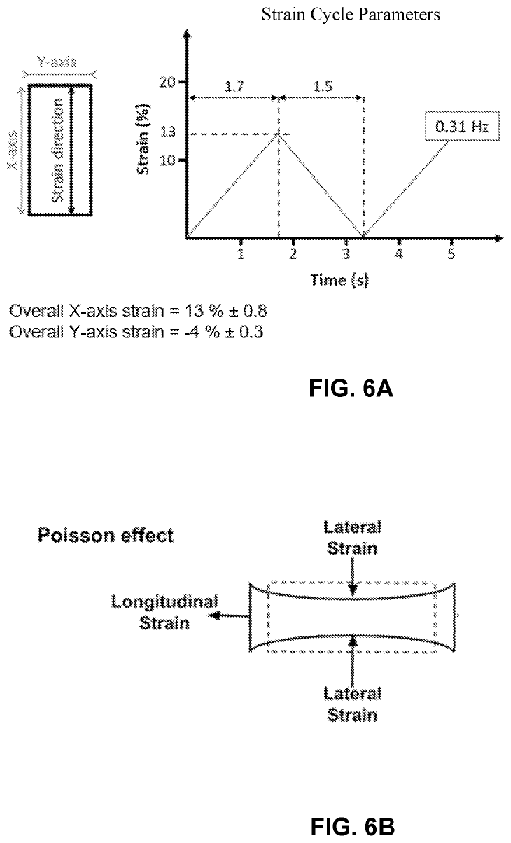

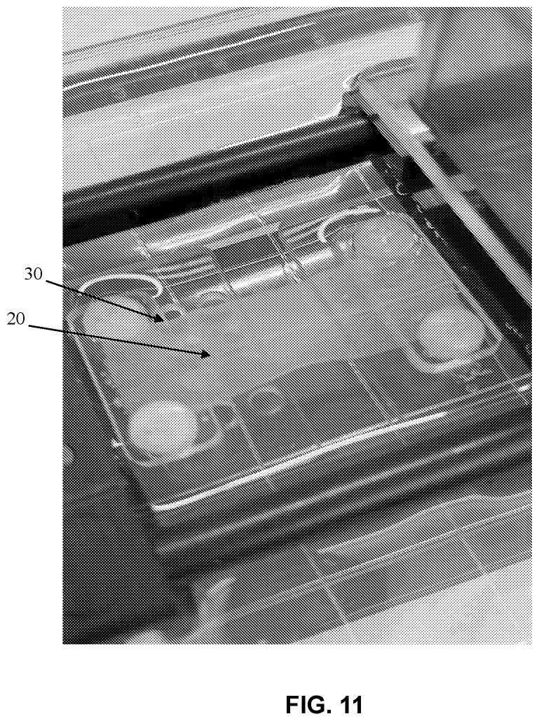

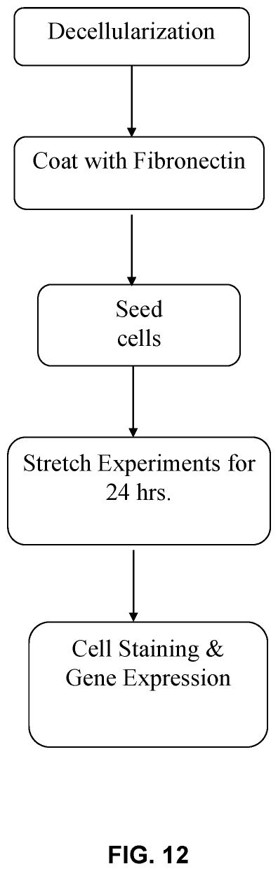

FIG. 6A is a graphical representation of cyclic strain that can be achieved with the actuator of the instant device. As desired, the magnitude, rate, frequency of strain are adjusted. For example, physiologically relevant parameters may be selected, including to match lung elongation during breathing, heart stresses/strains, blood vessel stress/strain, etc. FIG. 6B summarizes the longitudinal strain and the corresponding lateral strain generated due to the Poisson effect. FIG. 11 is a photograph showing the 3D scaffold 20 supported by stretchable substrate 30.

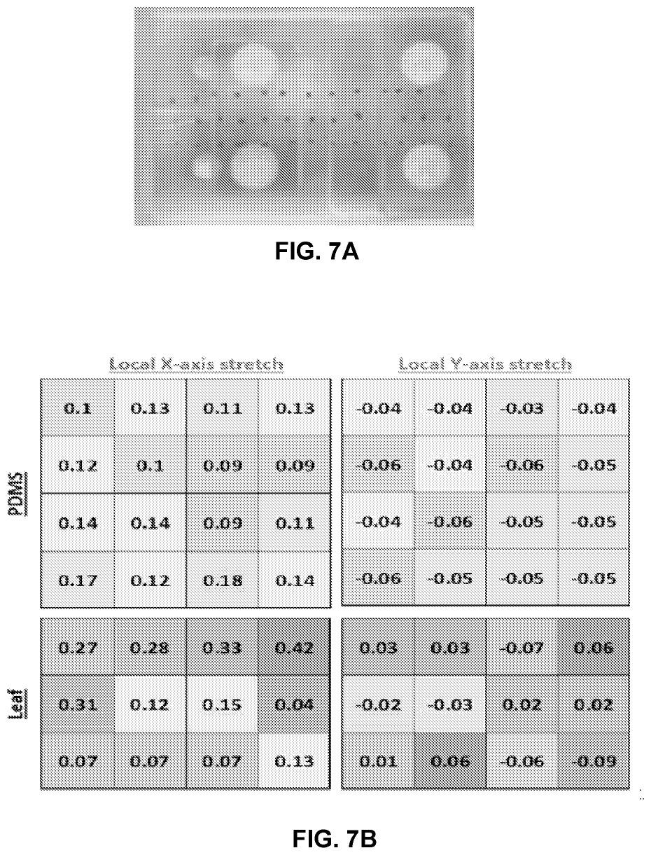

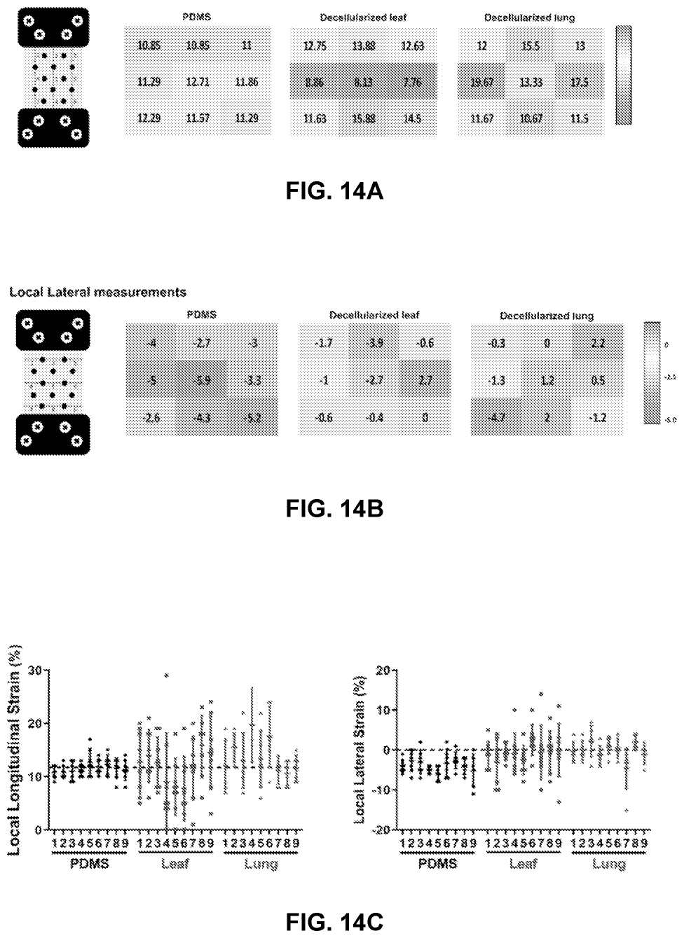

The characterization of the stretching and leaf scaffold elongation is provided in FIGS. 7A and 7B and 14A-14C. The Characterization Protocol as shown in FIG. 7A is: Several dots are placed on the biomaterial (cut in the shape of a typical leaf scaffold) equidistantly spaced between the two clamps in the clamping system. Then, videos of the biomaterial stretching (taken at 1080p resolution and 30 frames per second) are processed using imaging software, and analyzed using Image J to measure the displacement of each dot from its original position during the stretch.

### Example 3: Biological Response to Mechanical Stretch

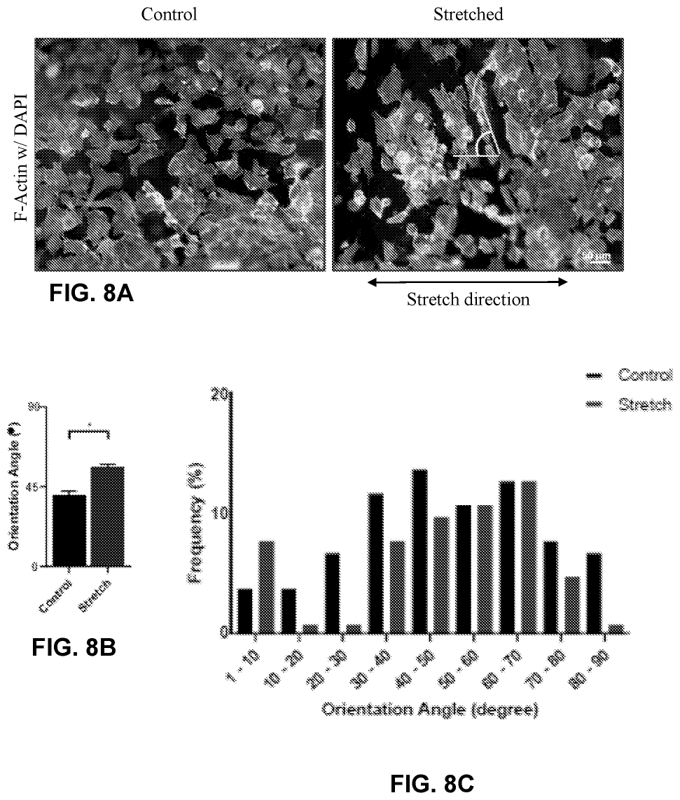

FIGS. 8A-10C, 13A-13C, and 15A-15I summarize biological response to cyclic stretch, including changes in morphology (including cell alignment), gene expression and protein expression, without adversely impacting cell viability.

The stretch device induces mechanical strain at physiologically relevant levels. Decellularized spinach leaf scaffolds is a proof of concept that 3-D scaffolds are capable of being stretched with the stretch device and that cultured cells present on stretched scaffolds respond to the mechanical stress.

This example illustrates that the stretch devices provided herein induce mechanical strain at physiologically relevant levels. Decellularized spinach leaf scaffold provides proof-of-principal that three-dimensional scaffolds can be supported by the stretchable substrate and reliably stretched via stretch of the stretchable substrate. Cells that are, in turn, supported by the three-dimensional scaffold are stretched and respond, biologically and physically, to the mechanical stress.

### Example 4: Stretching Decellularized Plant Scaffold Demonstrates a New Application for Plant-Based Biomaterials

The need for new biomaterials to provide effective scaffolding in tissue engineering (TE) has recently drawn attention to decellularized plant-based sources. Vegetal tissues innately possess key features of biological substitutes such as three-dimensional structure, biocompatibility and a highly interconnected porosity which promotes cell infiltration and facilitates nutrient/waste diffusion. Uniquely, plant leaves also provide a continuous network of vascular bundles which taper and branch similar to mammalian vasculature. Additionally, plant-based scaffolds are cost-effective and sustainably sourced. Provided herein are experiments validating this biomaterial and confirming its suitability for clinical applications.

As the interaction between a cell and its surrounding microenvironment is critical to determining cell function, there is a need to investigate the mechanical properties of decellularized vegetal scaffolds and their interface with the host tissues. Initial studies have shown that several of these scaffolds have a stiffness within the range of solid human tissues (1-20 kPa), such as the heart, lung or brain. Decellularized spinach leaves were also found to have a maximum tangent modulus of 0.30 MPa, similar to that of decellularized human tissue (0.20-0.50 Mpa). Another example showed that palm fibers displayed an ultimate tensile strength of 10-50 MPa and a strain at failure of 3-7% comparable to tendons in human (25-50 MPa, ˜ 2.5%). Interestingly, we show that cells seeded on decellularized vegetal tissue displayed a modified behavior, including downregulated YAP/TAZ signaling, decreased proliferation rates and altered morphology leading to a different response to external stimuli when compared to cells grown on standard tissue culture flasks. Collectively, these observations demonstrate that common mechanical properties of decellularized plant-based scaffolds are compatible with those of human tissue and may induce a cell behavior more representative of the in vivo cellular physiology than standard approaches.

However, certain tissues face additional and specific biomechanical constraints such as shear stress and/or cyclic strain. The latter has been a main component for lung TE where the bioreactor and biomaterial must be able to reproduce the forces generated by the ventilation. During normal human breathing, lung tissue is subject to a constant cyclic strain comprising of 10-20% elongation with a respiratory rate of 12-20 breaths per minute. This physiological stretching guides important cellular functions such as surfactant production, differentiation of type I and II cells, and control of cell proliferation.

Herein, we construct a custom-built device to stretch decellularized tissue in a cell culture environment and characterize the response of decellularized plant-based scaffolds to such deformation. Specifically, the goals of this example include: (1) determine if decellularized spinach tissue could support physiological lung-relevant cyclic mechanical strain and demonstrate that the response to this strain is similar to that of mammalian lung tissue, (2) establish that human alveolar basal epithelial cells seeded on the scaffold could sense and respond to this elongation and (3) investigate how cyclic strain could impact the drug response of these cells.

Vegetal and animal tissue decellularization: Baby spinach leaves were purchased from a local supermarket and decellularized. Briefly, the wax cuticle was removed with serial Hexane/1×PBS washes. Next, leaves were submerged in a 1% sodium dodecyl sulfate solution (SDS) for 48 hours on an orbital shaker. This was followed by a 10% sodium chlorite (bleach) solution for 12 hours to remove remaining debris. Leaves were then rinsed with deionized (DI) water to remove the residual chemicals.

Porcine lung tissue was obtained from Sierra Medical (California) and stored at −20° C. until use. Using a mandolin, 3 mm slices were cut and submerged in 2×PBS. Decellularization was performed on an orbital shaker in 5 cycles, alternating 1.8 mM SDS for 2 hours with a 15 min 2×PBS wash (O'Neill). Prepared samples were stored at 4° C.

Characterization of stretch device and strain measurement: To characterize the stretching properties of the custom-built device and decellularized tissue, the overall and local longitudinal and lateral elongation were measured. First, each material (polydimethylsiloxane (PDMS), decellularized spinach leaf and porcine lung tissue) were cut into a 3×1 cm section to fit into the device's clamping system. Before loading, a 3×3 matrix of reference points was drawn with a non-miscible marker on each of the rectangular scaffolds (FIG. 14A-14B). Once loaded into the device, the reservoir was filled with DI water and the stretching was initiated and recorded using an iPhone camera (iOS 14.8.1 software). Videos were then edited using a frame-by-frame analysis to extract images both at the original location and at the maximum elongation. These images were then analyzed using the Image J software to measure length changes between the reference points. The strain value (ε) was calculated using the formula (1):

$$ε ( \% ) = \frac{L - l}{l} \times 100 \qquad ( 1 )$$

Where L is the distance between the reference points at maximal strain and 1 the original length. Each material has been measured at least in triplicate.

The Poisson's ratio (v) of each material has been calculated using the following equation:

$$v = - \frac{{ε}_{l}}{{ε}_{t}} \qquad ( 2 )$$

Where εₗ and εₜ are the longitudinal and transverse strains, respectively, measured as previously described and calculated using Eq. 1.

Water retention assay: Plant scaffold tissue was dried for 48 hours at room temperature and the weight of the dried tissue was recorded. Next, the plant tissue was immersed in 2 mL of DI water for 1 hour at room temperature and weighed a second time after rehydration. The weight difference was considered as the amount of water retained by the tissue.

Mechanical Testing: Native and decellularized spinach leaves of known thickness were cut into a normalized rectangle shape and loaded into the grips of the mechanical tester. An Instron 3344 tester (Instron Corp., Norwood, MA) was used to uniaxially stretch the leaves. Leaves were stretched at a constant strain of 5 mm/mm until failure. Maximum tangent modulus, ultimate tensile strength, and strain at failure were calculated. Maximum tangent modulus was established by fitting a line to the maximal sloped linear region of the stress-strain graph. Ultimate tensile strength and strain at failure were calculated from the generated stress-strain graphs.

Cell culture and stretching: Adenocarcinome human alveolar basal epithelial cells (A549) were obtained from ATCC (CCL-185) and maintained in F-12K medium supplemented with 10% Fetal Bovine Serum (FBS) and 1% Penicillin/Streptomycin at 37° C. with 5% CO₂ atmosphere. After decellularization, spinach leaf scaffolds were cut into 3×1 cm pieces to fit into the device and sterilized with UV irradiation (Stratalinker 2400, Stratagene) for 30 min. The scaffolds were then incubated with a 1 μg/mL fibronectin solution (Sigma, F2006 2 MG) diluted in A549 medium and incubated overnight. The next day, the fibronectin solution was removed, and scaffolds were washed 3 times with complete medium. A549 cells were then seeded on the scaffolds using round inserts which were placed in the center of each sterilized scaffold to promote attachment overnight.

The disassembled stretch device was sterilized with 70% ethanol followed by UV irradiation for 30 min and then re-assembled in a sterile biosafety cabinet. Cell-seeded scaffolds were mounted and secured with 0.635 cm polycarbonate screws into the clamping system of the device. The clamping system with the scaffold was then placed into the reservoir device filled with complete medium to submerge the leaf. For the non-stretched control scaffolds, cell-seeded scaffolds were secured in a non-motorized control device with the same clamping system and filled with the medium. All experiments have been stopped after 24 h of stretching.

Immunofluorescence Staining and Microscopy: Cells were seeded on the decellularized spinach leaf scaffold at 120,000 cells/cm². After 24 hours under stretch conditions, the scaffolds were removed and fixed with 4% paraformaldehyde (PFA) in PBS for 5 min and cell membranes permeabilized with 0.1% Triton X-100/PBS for 5 min. Samples were then blocked for 30 min at room temperature in 1% BSA/PBS and immunostained with primary antibody YAP (sc-101199, Santa Cruz, 1/100) diluted in 1% BSA/PBS for 1 hour followed by Cy-3 conjugated anti-mouse IgG (115-165-062, Jackson ImmunoResearch, 1/400) or Alexa Fluo 488-conjugated Phalloidin (#A12379, ThermoFisher, 1/100) and counterstained with DAPI. Images were obtained using a Zeiss Axio Imager M2 epifluorescent microscope with a Zeiss AxioCam MRm camera using ZEN 4.5 software. The Nuclear/Cytosolic ratio of YAP was assessed by measuring the average intensity of a region of the nucleus and a region with equal size in the cytosol immediately adjacent to the nuclear region using Image J. The corresponding DAPI staining image was used to delimit nuclear versus cytosolic regions. A minimum of 300 cells were imaged from each of three independent experiments

Nuclear Morphology Analysis: The orientation angle of each cell nucleus was measured from 0-90° with respect to a line parallel to the x axis of the image using the Image J angle tool. To determine nuclear area, the Image J freehand selection tool traced the outline of each cell prior to taking the measurement. To determine sphericity, the length and width measurements of each cell's nucleus were taken using the Image J straight tool. The sphericity (Ψ) was then calculated using the equation (3).

$$Ψ = \frac{{{π}^{1 / 3} ( 6 Vp )}^{2 / 3}}{Ap} \qquad ( 3 )$$

Where Vp is the volume

$$V = \frac{4}{3} {π ( \frac{{r}_{a} + {r}_{b}}{2} )}^{3}$$

of the nucleus and Ap is the surface area A=4πrₐr_{b} of the nucleus with rₐ being the radius of the longitudinal axis and r_{b} being the radius of the lateral axis. A minimum of 300 nuclei were measured from each of three independent experiments.

Real-Time Quantitative Reverse Transcription PCR (qRT-PCR): 120,000 cells/cm² were seeded on the control and stretched vegetal scaffolds and incubated overnight at 37° C./5% CO2. After 24 hours, cells were lysed and RNA was extracted using RNAspin Mini kit (GE Healthcare, 25-0500-71) according to the manufacturer's recommendation and as previously described. RNA quantification was performed using an Epoch microplate spectrophotometer (BioTek Instruments). RNA was stored at −80° C. until further use.

For qPCR, 100 ng of total RNA was first used to generate cDNA using QuantiTect Reverse Transcriptase kit (Qiagen #205310) according to manufacturer's instructions. Briefly, the reaction was incubated at 42° C. for 2 minutes to eliminate residual genomic DNA and then placed immediately on ice. After addition of the reverse transcription mix, the reaction was placed at 42° C. for 15 additional minutes and stopped by incubation at 95° C. for 3 min before proceeding to PCR. QuantiNova SYBR Green PCR reactions (Qiagen #208252) were then carried out on a 96-well plate format using a Stratagene Mx3005P (Agilent Technologies, Inc.). Relevant primers are used.

Cycling parameters were 2 min at 95° C. for initial activation, followed by 40 cycles of denaturation at 95° C. for 5 s and combined annealing and extension at 60° C. for 10 s. Melting curves were automatically generated, ranging step-wise from 60 to 95° C. Data was collected by MxPro qPCR Software (Agilent). Values were normalized with glyceraldehyde-3-phosphate dehydrogenase (GAPDH) and analyzed according to the ΔΔCt method (Livak).

Calcium Assay and Image Analysis: Cells were seeded on the decellularized spinach leaf scaffold at 120,000 cells/cm². After 24 hours under stretch conditions, the medium was replaced with Fluo-4Direct calcium solution (1:1 solution of reagent to complete medium) for 1 hour (Fluo-4 Direct Calcium Assay, F10472, ThermoFisher). Scaffolds were then removed from their device, fixed with 4% PFA and counterstained with DAPI. Images were acquired using a Zeiss Axio Imager M2 epifluorescent microscope and ×63 oil-immersion objective lenses with a Zeiss AxioCam MRm camera using ZEN 4.5 software. To quantify Ca²⁺ level, in the GFP channel, the maxima intensity was measured using Image J and reported as a fluorescence intensity level per cell for each slice. A total of at least 100 cells for each of three independent experiments were quantified.

Masson's Trichome Immunohistochemistry Analysis: After 24 hours of stretching, samples were fixed with 4% PFA for 15 min and incubated in 70% ethanol overnight. Samples were further dehydrated using an alcohol gradient, cleared with xylene and embedded in paraffin blocks using an automated tissue processor system (Leica). Next, histological sections (4 μm thickness) were prepared from the paraffin blocks using a microtome. Slides were then cleared in xylene and rehydrated before staining with Masson's trichrome according to standard procedures. Samples were imaged using an optical microscope to assess the deposition of collagen in the tissues. Tissue processing and staining was performed.

Statistical Analysis: Statistical analyses were performed using GraphPad Prism software (GraphPad, version 9.3.1). Statistical comparison between two groups was done with the Student's/test or Mann Whitney test depending on whether or not the datasets passed the normality as assessed by the D'Agostino and Pearson test. All data was reported as mean with SEM unless otherwise noted, and p-value <0.05 was considered significant.

### Results:

Custom-built device stretches tissue within physiological values of normal human breathing: In order to stretch decellularized tissue to simulate the mechanical motions observed in normal human lung breathing, a custom-built stretch device was constructed (see, e.g., FIGS. 1A-1D). The experimental device was designed to meet a number of requirements and considerations compatible with biological parameters and applications. First, it must be biocompatible to support cell growth while being able to withstand sterilization protocols (UV+ethanol washes). It must also be of small size to minimize the volume of medium and cell culture reagents and to fit into a standard cell culture incubator. Once in the incubator, the device needed to be compatible for use in a cell culture environment where it will be exposed to heat and humidity to maintain cell viability.

As illustrated in FIG. 1C, the device may comprise: motor (1), which turns the cam (2) allowing the extension of the springs (3) which are attached to the slide mechanism (4). An arm attached the slide mechanism to the clamping system (5) which secured the leaf tissue. The clamping system was mounted into the reservoir (6) containing cell culture medium.

The physiological elongation of a normal, healthy human adult lung during breathing is between 10-20% with a respiratory frequency of 0.16-0.33 Hz. To characterize the strain magnitude of the device, the length between the two ends of the clamping system was measured while at rest and then again when under strain. As shown in FIG. 6A, the cyclic strain generated by the device has a triangular waveform with the average strain amplitude of 11.7%. The frequency of the stretch is 0.3 Hz with each cycle lasting approximately 3.3 s with equal time (1.67 s) for elongation and releasing phases. Collectively, this data demonstrated that our custom-built device can successfully stretch tissue to mimic the physiological values of cycle frequency and elongation which are observed during breathing motion.

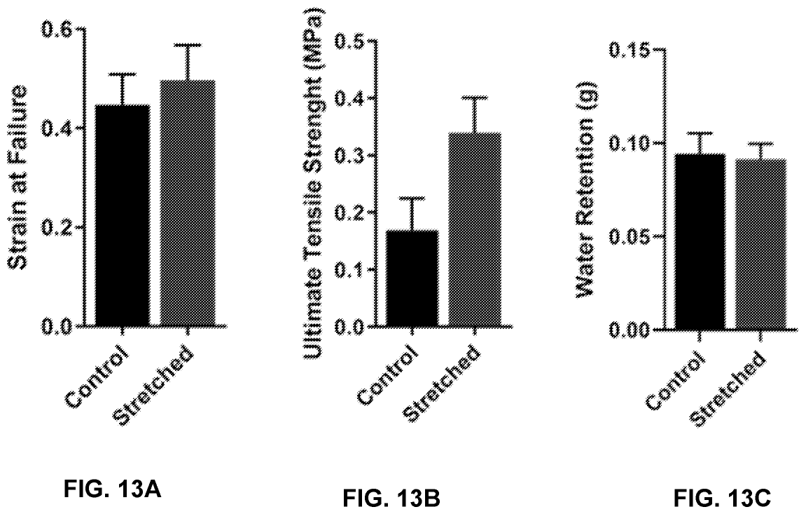

Scaffolds exposed to stretching conditions for 24 hours are structurally preserved: To assess the physical structure of the decellularized spinach leaf after exposure to the mechanical strain, scaffolds were first evaluated by mechanical testing for strain at failure and ultimate tensile strength (UTS). Results show the stretched scaffolds have a stain at failure of 0.496±0.07114, compared to 0.448±0.06102 for the unstretched controls (FIG. 13A). UTS found the stretched scaffolds to be 0.339±0.06192 MPa while the control was 0.169±0.05592 MPa (FIG. 13B). This indicates the scaffold polymers are not significantly altered by the elongation. In addition, the water retention properties of the two scaffolds were compared. Results showed the stretched and control scaffolds can retain a similar amount of water, 0.094±0.00834 g and 0.091±0.01093 g, respectively, (FIG. 13C) suggesting that the mechanical strain does not have a significant effect on the porosity of the scaffold.

Decellularized spinach leaves can be stretched and have mechanical strain response similar to that seen in mammalian lung tissue: Using our device, the behavior of decellularized spinach leaf scaffolds under strain was then characterized and compared with a standard elastomer polymer, polydimethylsiloxane (PDMS) and decellularized mammalian (porcine) lung tissue.

When a material is stretched (elongated), it may be subject to transverse compression/expansion in a phenomenon called the Poisson effect where a material contracts/expands in the direction perpendicular to the loading direction (FIG. 6B). This effect is defined as Poisson's ratio: the (negative) transverse strain to the axial strain. It provides a fundamental insight into the mechanical behavior of a material, typically generating a positive value between 0-0.5. Incompressible materials such as rubber have a value close to 0.5 while compressible materials like cork have a value close to 0. Scaffolds with zero Poisson's ratio have been suggested to be more suitable for emulating the behavior of native tissues and accommodating and transmitting forces to the host tissue site during wound healing (or tissue regrowth).

Thus, we first measured the bulk lateral strain of the three materials and calculated Poisson's ratio. Results showed the PDMS material had a ratio of 0.40 while the decellularized leaf and lung tissues were found to have a ratio of 0.12 and −0.01, respectively.

The Poisson's ratio for PDMS is consistent with previously published reports and demonstrates the incompressibility nature of this material. Interestingly, the decellularized leaf scaffold displayed a Poisson's ratio closer to zero and that of the decellularized lung tissue, suggesting that such a scaffold could be mechanically more suitable to mimic native tissue than standard PDMS.

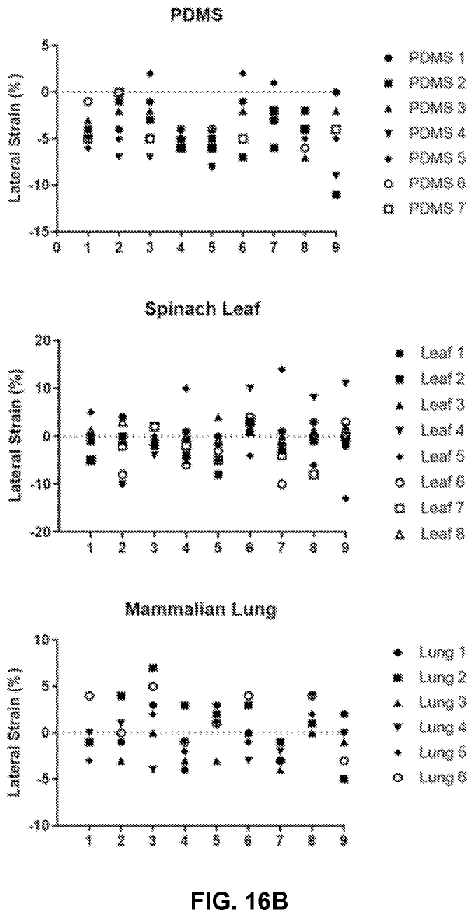

We next compared the local longitudinal and local lateral strain values of the three materials. To do so, we generated a 3×3 grid on each biomaterial and measured the longitudinal strain or lateral strain within each cell (FIG. 14A). As seen in FIG. 14B, the local longitudinal strain values of the PDMS are relatively close to the average value of 11.7% (between 10.85-12.71%) while the decellularized leaf and lung scaffolds showed greater variation, ranging from 7.76-15.88% and 10.67-19.67%, respectively (FIG. 14C). This was not surprising given the heterogeneous nature of the tissues which are purposefully patterned with extracellular matrix proteins or a branching vascular network. Interestingly, we confirmed that this variability is not correlated to an intrinsic pattern within each sample, but rather can be attributed to independent, local differences (FIGS. 16A-16B).

The local lateral strain, also known as a transverse compression (or expansion), was similarly measured on the 3×3 grid and measurements were performed perpendicular to the stretch direction. If compression is observed, then the lateral strain value is negative as primarily seen with PDMS, −5.9-−2.6% (FIG. 13C). The decellularized leaf scaffold had both positive and negative values, from −3.9-2.7% and the decellularized lung tissue similarly ranged from −4.7-2.2%. Not surprisingly, the decellularized spinach leaf and mammalian lung tissue scaffolds displayed a higher variation than that of the PDMS due to the homogenous nature of the PDMS and heterogeneous properties of the tissue scaffolds. In addition, the PDMS local lateral strain values were to be lower than those observed with the leaf and tissue scaffolds, confirming the transversal compression of this material during stretching and its higher Poisson's ratio.

Overall, we demonstrated that the device could support stretching of the decellularized vegetal tissue and that the vegetal tissue was able to be stretched by the device to an 11.7% longitudinal strain to meet our desired parameters. Additionally, the characterization of the decellularized leaf scaffold under mechanical strain displayed a high heterogeneity and local variability as observed in mammalian decellularized lung tissue.

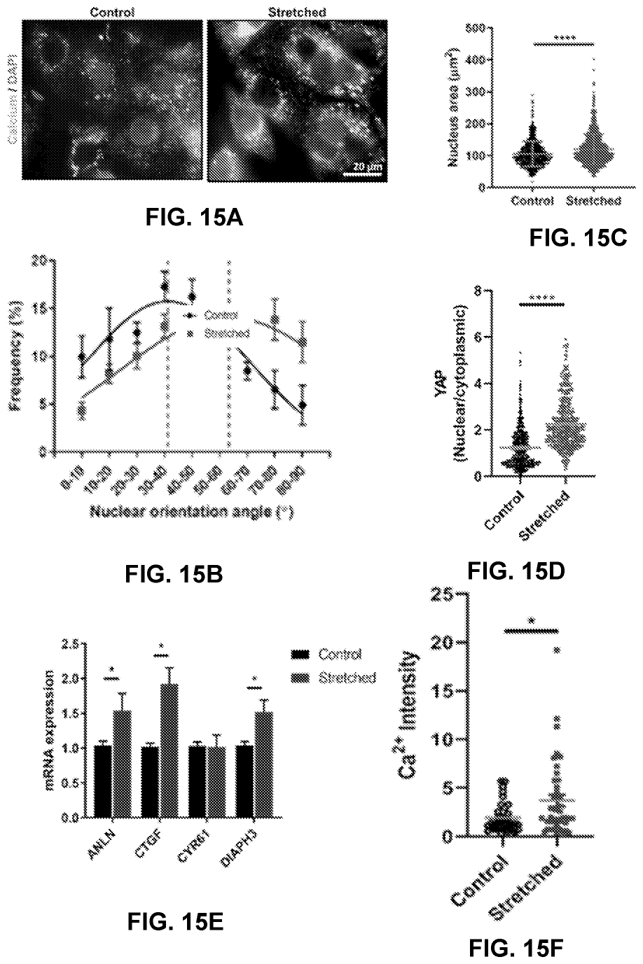

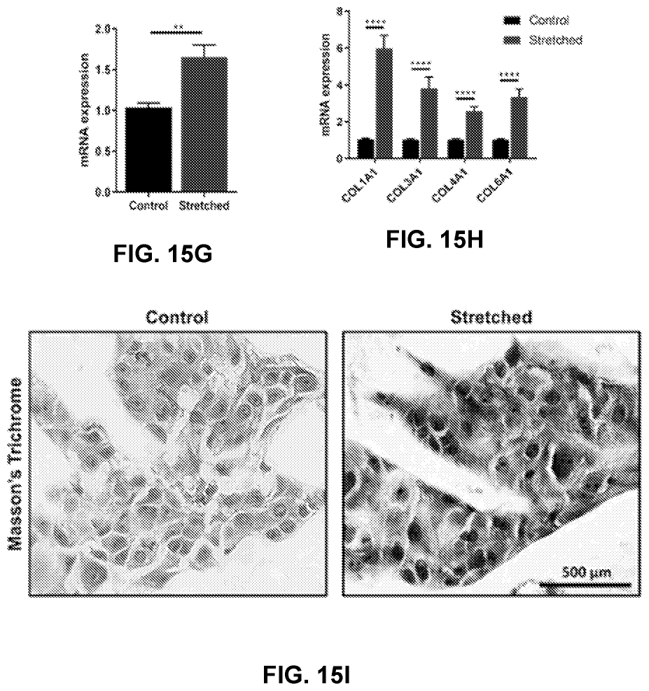

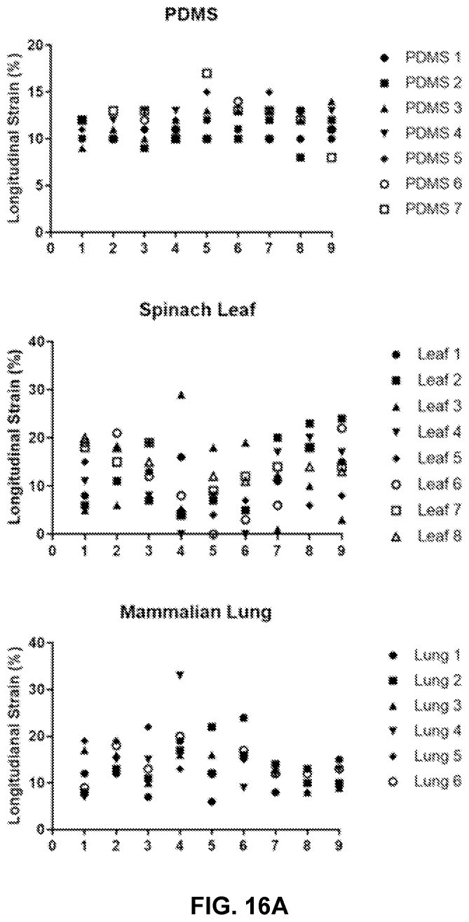

Cells seeded on decellularized spinach leaf scaffolds can sense and respond to the mechanical strain: To assess if the cells seeded on the decellularized spinach leaf scaffold being stretched by our device can sense this mechanical deformation, we seeded human epithelial alveolar lung cells (A549) onto the scaffold and assessed their response to stretched conditions for periods of 24 hours. It has been documented that cellular morphology can be modified by mechanical strain, including a reorganization of cytoskeleton and perpendicular alignment to the stretch direction, a larger nucleus area and a modified nucleus aspect ratio. As seen in FIG. 8A, the stretch conditions caused A549 cells to orient themselves perpendicular to the direction of the stretch. In the non-stretched control group, however, cells were found to be randomly oriented. Measurement of the nuclear orientation angle of the cells confirmed this observation. Cells on the stretched scaffold displayed a mean angle of 63.3° compared to the stretch direction, while those seeded on static scaffold showed a nuclear angle of 41.2° (FIG. 15B). This stretch-induced re-orientation of the nuclei was associated with a measured increase in the nuclear area, averaging 120.7 μm² compared to 106 μm² for the control group (FIG. 15C). Significant differences in nuclear sphericity, however, were not seen. This evidence demonstrates that A549 lung cells seeded on stretched scaffold displayed modified cellular morphology similar to that of cells under strain constraints.

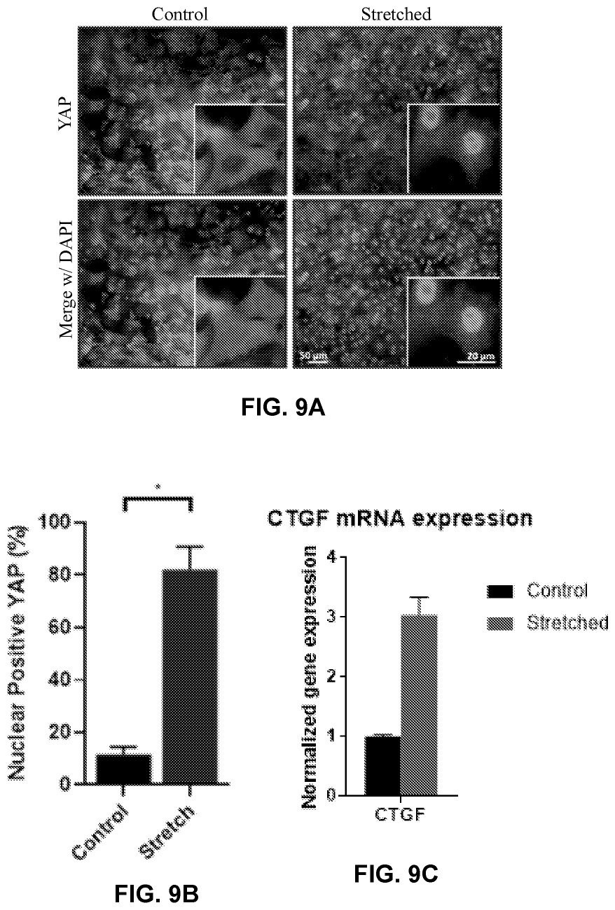

Moreover, cells sense and respond to physical and mechanical cues in their surrounding environment through signaling pathways in a phenomenon known as mechanotransduction. Hippo pathway signaling proteins YAP (yes-associated protein 1) and TAZ (transcriptional coactivator with PDZ-binding motif) are mechanosensitive transcriptional co-regulators. When inactivate, YAP is phosphorylated and remains in the cytoplasm. However, upon response to mechanical cues, the YAP/TAZ complex is translocated to the nucleus where it activates multiple target genes involved in cell proliferation, differentiation and migration. Therefore, to investigate the cyto-nuclear shuttling of the YAP/TAZ complex, we used immunofluorescence staining to visualize the location of YAP. Under strain conditions, YAP was found to be predominately nuclear. In contrast, YAP remained primarily in the cytoplasm of the unstretched control (FIG. 9A). These observations were confirmed by the quantification of the mean intensity of nuclear and cytoplasmic YAP (FIG. 15D). To assess if the nuclear translocation of YAP was associated with functional activity, we also quantified the expression of known target genes of YAP. Related genes ANLN, CTGF and DIAPH3 were each found to be significantly overexpressed in cells exposed to strain conditions compared to the un-stretched control (FIG. 15E). Taken together, this data demonstrated that the YAP/TAZ pathway was upregulated in cells seeded onto stretched scaffolds. Further, mechanical strain is known to stimulate the release of intracellular calcium through stretch-activated calcium channels. Using a Fluo-4 probe, we showed that cells seeded on stretched scaffolds had a significantly higher fluorescence intensity than cells on static scaffolds, suggesting an increase in intracellular calcium level under strain constraint (FIGS. 15A and 15F). To confirm this observation, calmodulin (CALM1) gene expression was also quantified. CALM1 is a prototypical and versatile cellular calcium sensor (Chin and Means, Zhang and Zhang Structure 2012), activated by the increase and binding of intracellular calcium that trigger changes in its conformation to form complexes with a diverse array of target proteins. Results showed that the CALM1 gene was significantly upregulated in cells seeded onto stretched scaffolds compared to control (FIG. 15G).

Finally, it has been demonstrated that under mechanical stretch, lung cells display an elevated level of collagen deposition. Therefore, we performed gene expression analysis on four of the major types of collagen types which are known to be secreted by lung cells. Results showed collagen genes COL1A1, COL3A1, COL4A1 and COL6A were significantly upregulated in cells seeded onto scaffold exposed to strain conditions (FIG. 15H). To assess if collagen production was indeed increased, Masson's trichrome histological staining was performed and revealed the prominent blue color of collagen fibers on cell-seeded stretched scaffolds (FIG. 15I) confirming an increase in collagen secretion.

Taken together, this example demonstrates that epithelial alveolar lung cells seeded on the decellularized spinach leaf can respond to the elongation of this scaffold based upon changes in cell morphology, mechanotransduction signaling pathways, calcium release and collagen deposition, suggesting that they are stretched and even able to sense this physiological-lung relevant mechanical strain.

Mechanical strain modifies cell drug response: As we established that the cells respond to the mechanical strain, we also confirm the drug response of the lung epithelial cells (A549) cells is modified by the physiological stress/strain through the application of drug and measuring, after 72 hours, differences between static and stretched cells for cellular proliferation/mitotic events/apoptotic events.

Discussion: Many anatomical areas of the human body stretch, bend or flex which contributes to enhanced function. This is observed, for example, during human breathing where the lungs expand to facilitate gas exchange. Upon inhalation, the diaphragm muscle contracts, enlarging the chest cavity and generating a vacuum that fills the lungs with air. When the muscle relaxes, the lungs reduce in volume and air is expelled. This cyclic expansion/contraction is critical to the exchange of carbon dioxide waste for fresh oxygen which is then transported via the bloodstream to cells within the human body. In this example, we demonstrated a new application of decellularized plant tissue by stretching this biomaterial to mimic the breathing patterns found in the human lung. Human lung cells sense and respond to this mechanical strain and have an altered drug response. In this way, our biomaterial has an enhanced utility which can be more similar to the in vivo condition.

To demonstrate this new application, we first built a custom device to secure and apply mechanical strain to the tissue. Many commercially available systems are available to stretch cells seeded on a fixed membrane inside of a cartridge. However, our device is unique in that it can clamp and stretch cell-seeded three-dimensional scaffolds that better mimic biological tissues. We tailored the device to meet the physiological relevant parameters of the breathing of a human adult (longitudinal elongation between 10-20% with a respiratory frequency of 0.16-0.33 Hz). We demonstrated that the device stretched the tissue with 11.7% strain at a frequency of 18 stretches/minute. However, this device can be tuned to different physiological parameters to apply this same mechanical principle to other anatomical areas such as the heart, arterial blood vessels, skin or tendon. The exchangeable motor can be modified to meet the appropriate frequency of strain needed while tuning the off-centered cam alters the percentage of applied strain. Further, it was important that we also developed a device that was small in size so that many could fit in a cell culture incubator and withstand the heat and humidity conditions therein. Thus, this scalable device brings a new application for tissues and is adaptable for numerous applications.

We further characterize the strain behavior of the decellularized vegetal tissues and compare those results to that of a well-known synthetic material, PDMS, as well as a natural material, decellularized porcine lung tissue. The results were revealing; PDMS was found to have a Poisson's ratio of 0.4, similar to previously established values and demonstrating the high incompressibility of this material. In contrast, the decellularized vegetal and porcine tissues were found to be highly compressible displaying values much closer to 0 (0.12 and −0.01, respectively). As Poisson's ratio is a universal way to compare structural performance of a material, this measurement shows the porcine lung tissue does not transversely deform upon in response to the mechanical strain where the PDMS exhibits considerable contraction under the same strain force. The decellularized plant scaffold displayed a value similar to the porcine lung tissue, demonstrating low transverse contraction which reinforces the favorable use of this material as a biological substitute. Many proposed biomaterials are engineered or tuned to display an ideal Poisson's ratio by altering internal porosity. However, this is an innate property of the decellularized spinach leaf scaffold that does not require modification. Other decellularized plant tissues can be tested to confirm whether this property is similarly observed. Additionally, we expect specific Poisson ratios of decellularized plant tissues may be altered by varying the decellularization method. This provides another means of fine-tuning scaffold mechanical property to better mimic the in vivo environment.

Interestingly, when we compare the local strain of the decellularized plant tissue to that of decellularized porcine lung tissue we found that these tissues had several similarities. Both reveal the heterogeneity of the materials.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Lacombe, J., Harris, A. F., Zenhausern, R., Karsunsky, S., & Zenhausern, F. (2020). Plant-Based Scaffolds Modify Cellular Response to Drug and Radiation Exposure Compared to Standard Cell Culture Models. Frontiers in bioengineering and biotechnology, 8, 932. doi.org/10.3389/fbioe.2020.00932
2. Joshua R. Gershlak et al. (2017), Crossing kingdoms: Using decellularized plants as perfusable tissue engineering scaffolds, Biomaterials, 125:13-22. doi.org/10.1016/j.biomaterials.2017.02.011.
3. Harris, A. F., Lacombe, J., & Zenhausern, F. (2021). The emerging role of decellularized plant-based scaffolds: Challenges and Perspectives. Int. J. Mol. Sci. 22 (22): 12347.
4. Wang, W. H., Hsu, C. L., Huang, H. C., & Juan, H. F. (2020). Quantitative Phosphoproteomics Reveals Cell Alignment and Mitochondrial Length Change under Cyclic Stretching in Lung Cells. Int. J. Mol. Sci., 21 (11), 4074. doi.org/10.3390/ijms21114074
5. Pugin, J., Dunn-Siegrist, I., Dufour, J., Tissières, P., Charles, P. E., & Comte, R. (2008). Cyclic stretch of human lung cells induces an acidification and promotes bacterial growth. Am. J. Respir. Cell Mol. Biol., 38 (3), 362-370. https://doi.org/10.1165/rcmb.2007-0114OC

1. A device for stretching a three-dimensional scaffold under cell culture conditions comprising:
a stretchable substrate for receiving the three-dimensional scaffold;
a stretchable substrate holder configured to connect to the stretchable substrate;
an actuator operably connected to the stretchable substrate holder to exert a cyclic stretch on the stretchable substrate and periodically stretch the three-dimensional scaffold connected to the stretchable substrate; and
a sample holder configured to immerse the three-dimensional scaffold in a tissue culture media and expose the three-dimensional scaffold to cell culture parameters for ongoing viability of living cells supported by the three-dimensional scaffold.

2. The device of claim 1, wherein the three-dimensional scaffold comprises a natural scaffold or an artificial scaffold.

3. The device of claim 1, wherein the stretchable substrate comprises a polymer, a cellulosic film, a thin piezoelectric material, a bi-metallic memory material, or a decellularized tissue.

4. The device of claim 1, further comprising a biocompatible layer positioned between the stretchable substrate and the three-dimensional scaffold.

5. The device of claim 1, wherein the actuator comprises a power supply, a cam, and a gearbox operably connected to generate a mechanically driven translational motion of a portion of the stretchable substrate holder connected to the stretchable substrate.

6. The device of claim 1, wherein the actuator comprises one or more of an electrical motor, an electrical vacuum pump, a fluidic pump; and/or an electromagnetic actuator.

7. The device of claim 1, wherein the actuator is a uniaxial, bi-axial, multi-axial or radial actuator.

8. The device of claim 1, wherein the actuator provides a stretch up to 100%, including up to 20% stretch, for a time period up to 2 months under the cell culture parameters.

9. The device of claim 1, further comprising an electronic controller connected to the actuator to provide a user-specified stretch protocol, wherein the user-specified stretch protocol includes a maximum strain magnitude, strain rate and/or time-course.

10. The device of claim 1, further comprising a power source that is a battery.

11. The device of claim 1, further comprising a cell culture controller to control the cell culture parameters around the stretchable substrate, including cell culture parameters that are temperature between 32° C. and 40° C., a CO₂ level of between 4% and 6%, an O₂ level of between 2% and 21%, and/or a humidity of between 80% and 98%.

12. The device of claim 1, wherein the device has a device geometric parameter configured to position at least the stretchable substrate and three-dimensional scaffold into a cell culture incubator that provides the cell culture parameters.

13. The device of claim 1, further comprising a sensor for monitoring one or more of the cell culture parameters.

14. The device of claim 1, wherein the three-dimensional scaffold comprises a decellularized plant tissue.

15. A method of periodically stretching a three-dimensional scaffold, the method comprising the steps of:
connecting a stretchable substrate to the stretchable substrate holder of claim 1;
attaching the three-dimensional scaffold to the stretchable substrate; and
actuating the actuator to exert a periodic force on the stretchable substrate by the actuator, thereby periodically stretching the three-dimensional scaffold.

16. The method of claim 15, wherein the exerting step is for a time period ranging from minutes to weeks.

17. The method of claim 15, further comprising the step of culturing one or more biological cells supported by the three-dimensional scaffold.

18. The method of claim 17, wherein the actuating step is selected to produce a desired biological response parameter from the one or more biological cells, the response parameter selected from the group consisting of: gene expression; protein expression; metabolite secretion; mutation; cellular microvesicle release; phenotype expression; alignment; and cell proliferation.

19. The method of claim 15, wherein the three-dimensional scaffold comprises a decellularized scaffold from a vegetal or an animal tissue (e.g., extracellular matrix such as collagen, elastin and/or glycosaminoglycan); artificial scaffold made with a synthetic material (e.g. silicon, polylactic-coglycolic acid, polyurethane, poly(glycerolsebacate), polyacrylamide, etc.) or natural material (e.g. plant protein (e.g. soy, zein, wheat glutenin, etc.), plant polysaccharides (e.g. cellulose, pectin, starch, etc.), lignin, plant extracts, alginate, collagen, gelatin, hyaluronan, fibrin, chitosan, etc.); or a polymer.

20. The method of claim 15, wherein the actuating step comprises a maximum bulk stretch of the three-dimensional scaffold of up to 100%, including between 5% and 25%, at a cyclic stretch of between 1 cycle per minute and 200 cycles per minute.

21. The method of claim 15, wherein the stretchable substrate has a uniform stretch distribution and the three-dimensional scaffold has a spatially varying stretch distribution.

22. The method of claim 15, further comprising the step of controlling a periodic force to obtain a desired biological response of biological cells cultured in the periodically stretched three-dimensional scaffold.
